(19) United States
(12) Patent Application Publication
(10) Pub. No.: US 2026/0295413 A1
(43) Pub. Date: Oct. 1, 2026

(54) SYSTEMS AND METHODS FOR PRESENTING VISUAL, AUDIBLE, AND TACTILE CUES WITHIN AN AUGMENTED REALITY, VIRTUAL REALITY, OR MIXED REALITY GAME ENVIRONMENT

(71) Applicant: Strolll Limited, ALDRIDGE, WALSALL (GB)

(72) Inventors: Daphne Jeanne Geerse, Haarlem (NL); Melvyn Roerdink, Gulpen (NL); Ben Wain, Shrewsbury (GB); Jorgen Ellis, London (GB)

(21) Appl. No.: 19/100,898

(22) PCT Filed: Aug. 1, 2023
(86) PCT No.: PCT/IB2023/057777

§ 371 Date: Feb. 3, 2025

(30) Foreign Application Priority Data
Aug. 1, 2022 (GB) 2211189.2

(51) Int. Cl. A63F 13/533; A63F 13/215

(52) U.S. Cl. CPC A63F 13/533; A63F 13/215; A63F 2300/8082

(57) ABSTRACT

An aspect of the invention provides an augmented reality system for presenting cues to a subject who experiences gait impairment, wherein the augmented reality system comprises a display for providing an augmented reality overlay over a subject's field of vision and wherein the augmented reality system is arranged and adapted to: i) generate for display to the subject an interactive media asset; ii) determine the subject's current position; iii) generate for display to the subject a first virtual asset; iv) determine the position of the first virtual asset relative to the subject's current position; v) generate for display to the subject at least one cue which acts to prompt the subject to step forwards and walk from the subject's current position to the position of the first virtual asset.

### FIELD

Aspects of the present invention relate to systems and methods for presenting visual, audible, and tactile cues within an augmented reality, virtual reality, or mixed reality game environment. Certain embodiments of the disclosure are related to systems and methods for modifying gait and providing gamified rehabilitation via augmented reality, virtual reality, or mixed reality headsets/glasses in individuals suffering from neurological impairments and conditions including, but not limited to, Parkinson's disease, Stroke or MS.

### BACKGROUND

Individuals suffering from neurological disorders such as (but not limited to) Parkinson's, Stroke, MS often have impaired gait, postural instability, increased risk of falls and reduced physical activity. Gait disturbances can be presented in several ways, from a reduced step/stride length, increase step width variability, slowed gait speed, difficulty initiating gait, or other debilitating gait impairment. To date, surgical and pharmacological intervention is not sufficient in helping a person to overcome these symptoms.

Gait impairment, postural instability and increased risk of falls negatively impacts mobility and independence and can cause significant emotional stress in a patient resulting in a reduced quality of life.

It has been observed that whilst people with neurological disorders may struggle with self-directed or autonomous movement such as walking, other activities which involve goal directed, or attention orientated movement may remain relatively unaffected by the underlying disease.

In response to this recognition, several experiments have been performed wherein external cues are provided to a person with Parkinson's disease, Stroke, MS or other disease to modify their gait pattern and improve their ability to walk. It is known to provide a pattern of coloured stripes on the floor which provide visual cues to the person or to provide an auditory rhythmic cue such as a metronome which a person will walk in time to the beat of. A physiotherapist working with a patient may, for example, use line markers on the floor which can act as a visual cue, or play a metronome or music with a regularly timed beat. The patient suffering from gait impairment is encouraged to walk on these visual cue points or in time to the beat of the metronome or music and this strategy has been found to be effective.

Whilst providing physical visual markers on the floor, or auditory rhythmic cues with metronomes or music has been found to be effective, it will be apparent that such an approach is relatively basic and time consuming as it requires a person to physically to set up the markers or auditory system, which both need to be personalised to an individual's gait parameters (step length, gait speed, step width variability etc) and then reset after the task is performed or the session is completed for the next patient. Furthermore, this approach assumes that all patients will respond in the same way to visual and auditory cues, however this is not the case. There is a high rate of heterogeneity both within and across neurological disorders such as Parkinson's disease or Stroke, and it has been proven that while one sub-set of visual cues e.g., Line Markers may be effective for one patient, they could be totally ineffective for another patient who instead benefits from visual obstacles to step over to achieve the same gait modifying effect. The same goes for auditory cueing, where a metronome may be effective for one patient, another patient may benefit more from the sound of footsteps on gravel.

Additionally, the gait modifying effects of individual cues have been shown to wear off over time, often referred to as habituation. To maintain the positive effect and gait modifying ability, cues need to be adapted over time and continuously changed to avoid habituation. For example, after 6 months of using the visual obstacles as a Cue, these could be changed to dinosaur footprints for a patient to step on, providing a completely new visual cue that helps a person to maintain the positive effect and avoid habituation.

Furthermore, traditional cueing methods assume that only a single person is seeking to navigate a particular space at any time either for directional walking or functional movement therapy. The current state of the art is difficult to utilise with multiple patients in the same room at the same time who likely have very different gait parameters such as step length or gait speed with each individual requiring different types or spacing of visual markers on the floor or a different auditory rhythmic soundtrack or bpm (audio speed). It will be appreciated in a situation where multiple people are seeking to navigate or conduct physical therapy in a particular space at the same time (for example, patients in a care home or hospital environment) then this conventional approach no longer works as it is too confusing.

To address this, the applicant has previously developed an augmented reality (AR) software application that is implemented through AR glasses/headset. Such an application provides a display for providing an AR overlay over a subject's field of vision which visually masks or obscures any hazards or unmapped areas which might distract, attract or confuse the subject and is able to display a library of images, visual cues which appear to the subject to be located in front of them and which act to prompt —in a goal-directed manner—the subject to step forwards and follow a determined safe or otherwise optimum path from the subject's current location to a desired destination.

Visual cueing applications using AR technology have been proven to have strong potential for modifying gait in person's living with gait impairment, postural instability, and increased fall risk as a result of neurological disorders such as (but not limited to) Parkinson's, Stroke, MS. Furthermore, it has been proven that, in isolation, visual cues work better than other cueing modalities (acoustic, tactile, etc) due to a strong sensorimotor coupling as well as a stronger action relevance or goal-directedness. AR technology enables a subject's environment to be mapped and visual cues placed on the AR overlay in action relevant locations. AR technology also enables auditory and tactile cues to be presented to a wearer.

Existing AR applications for modifying gait require a subject to activate the visual/auditory cues when required. This may be achieved by pressing a button, issuing a voice command, or making a gesture, for example. Manual activation of the visual cues may be difficult for some subjects who suffer from significant cognitive and/or motor impairment. For example, subjects experiencing tremors, monotonic soft/impaired speech and/or cognitive decline may not be physically able to activate visual/auditory cues using a physical control means or voice command. Consequently, visual cues/auditory cues may be difficult for a person to access or use during an active physical therapy session.

Using an AR headset, a subject's environment may be mapped to determine locations where there is free space and locations where there are obstacles or hazards. Based on this environmental data, a series of visual cues may be plotted between two or more points. For example, in a domestic environment, the environmental data may identify that there are certain obstacles between two virtual objects. Thus, visual cues may be presented along a defined path between the two objects that is optimised to avoid all detected obstacles such as furniture or other obstructions. The visual cues may be selected from a variety of different visual cue subsets accessed via a software library within the AR headset and pre-set to the individuals gait parameters, e.g.. visual objects to step on are calibrated to the users step length or the desired step length set by the patient's physical therapist.

Thus, the present invention seeks to provide an improved augmented reality system to provide gamified physical therapy for people with gait impairment resulting from neurological disorders by providing personalised, multi-sensory cueing which can be personalised to individuals from a library of visual/auditory/tactile cues within a virtual/augmented game environment. In particular, the present invention seeks to provide a reliable and tailored system and method for activating visual and/or auditory cues via an AR headset within a game environment to provide physical therapy. The present invention also seeks to provide a proactive (as opposed to reactive) system and method of activating visual and/or auditory cues in accordance with a user's real time needs within a virtual rehabilitation game environment.

### SUMMARY

Aspects of the present invention relate generally to an augmented reality system configured to generate visual, auditory, or tactile cues either in isolation, or as a combination of cues. It is well recognized that the use of cues (whether visual, auditory, or tactile) has the effect of offering goal-directed templates to bypass the defected brain areas associated with non-intentional automatic movement control to and mitigate the effect of gait impairment, postural instability and fall risk. This effect can be achieved through a series of therapeutic goal-directed activities such as the playing of simple games that require a subject to move between objects in order to perform a certain motor task. For example, in an augmented reality game, a subject may be required to move towards mole hills to stand on a holographic mole character. Without the aid of cues, a subject may find it difficult to move between the two mole hills within the augmented reality game or may not be able to play the game at the desired intensity level required by the therapist. Thus, the applicant has determined that there is a beneficial, therapeutic effect of generating visual cues for display within an augmented reality rehabilitation game environment that extend between the subject's present position and a location of a hologram in virtual space. In some embodiments, the visual cues may be configured to avoid any obstacles that are mapped in the physical world. In some embodiments cues will be personalised automatically to the individuals gait parameters. In some embodiments, cues will dynamically adapt to the user's real and gamified environments and movement. Furthermore, auditory and/or tactile cues may be presented to the subject in conjunction with, or in place of, the visual cues.

References to augmented reality, or AR, headsets herein refer to head mounted display devices that provide imagery to the subject through a display screen positioned in front of the subject's eyes. The display screen is transparent meaning that the subject can see both real world objects and virtual objects. The virtual objects may be manipulated to interact in a virtual space with real world objects. Images, text, video, animations, games, etc, may be generated for display and presented to the subject through an AR headset. One such example of an AR headset that may be used to implement embodiments of the invention is Microsoft Hololens 2. This device enables holographic images to be presented on a surface, i.e., floor or tabletop, and in free space that is only constrained by the field of view of the AR device.

One aspect of the invention provides an augmented reality system for presenting cues to a subject who experiences gait impairment, wherein the augmented reality system comprises a display for providing an augmented reality overlay over a subject's field of vision and wherein the augmented reality system is arranged and adapted to: i) generate for display to the subject an interactive media asset; ii) determine the subject's current position; iii) generate for display to the subject a first virtual asset; iv) determine the position of the first virtual asset relative to the subject's current position; v) generate for display to the subject at least one cue which acts to prompt the subject to step forwards and walk from the subject's current position to the position of the first virtual asset.

The present invention seeks to provide cueing assistance to subject's suffering from gait impairment through an “in-game” environment. By determining the position of a subject and the position of a virtual asset within a virtual space embodied within an augmented or mixed reality headset, at least one cue may be presented to the user to assist in walking between his/her current position and the determined position of the virtual asset. This has the beneficial effect of modifying and improving gait within an augmented reality or mixed reality environment where the subject is required to move between virtual assets that are not visible outside of the augmented reality or mixed reality headset.

In one embodiment at least one cue comprises a series of transverse bars, images or other visual cues which appear to the subject to be located on the ground in front of the subject.

In one embodiment the augmented reality display comprises a headset or glasses.

Use of a headset or glasses enables the subject to interact with the visual, and other, cues without using their hands to interact with a device. This is beneficial as it frees up the subject's hands to interact with virtual objects within the game environment and enables the subject to focus on the game content rather than the device that is presenting the content to the subject.

In one embodiment the augmented reality system measures and records gait parameters of the subject.

By measuring and recording gait parameters of the subject, visual, audio, and tactile cues may be presented to the subject based on a real time analysis of the subject's gait. For example, if the subject's gait is determined to have changed along the cued path, the length, frequency, colour, thickness, height, for example, of the cues may be varied accordingly. The effect of the variation of cues on the subject's gait may also be recorded with several different cue parameters being adjustable to determine the most appropriate cue to mitigate an observed gait impairment in any situation.

In one embodiment the augmented reality system is further arranged and adapted to: generate for display to the subject a second virtual asset; determine the position of the second virtual asset relative to the first virtual asset and/or subject's current position; and generate for display to the subject a series of transverse bars, images or other visual cues which appear to the subject to be located on the ground in front of the subject and which act to prompt the subject to step forwards and follow the transverse bars, images or other visual cues from the position of the first virtual asset to the position of the second virtual asset.

In a virtual and/augmented reality environment, a subject may wish to move between virtual objects that are displayed via the augmented reality headset. Such virtual objects may be randomly generated to appear either inside or outside of the subject's field of vision. In the event of an onset of gait disturbance, the subject may find it difficult to navigate from the first visual object to the second visual object even if presented with simple navigational tools such as directional indicators or instructive prompts. Therefore, the display of visual cues between virtual objects is highly advantageous to enable the subject to move therebetween. In addition, auditory and tactile cues may be used firstly in combination with visual cues to augment the effect on the brain. Auditory and/or tactile cues may also be used without visual cues in certain circumstances.

In one embodiment the series of transverse bars, images or other visual cues is displayed in different colours depending on the subject's current position relative to the position of the series of transverse bars, images, or other visual cues.

In a game environment, it may be advantageous to communicate information to the subject using the visual cues. For example, the user's position may be matched to a target position through use of visual cues of differing colour. In one embodiment, the visual cues may be presented in red if the subject is significantly off course, orange if the subject is slightly off course, and green if the subject is on course. Instead of visual cues, auditory and tactile cues may be implemented instead.

In one embodiment the visual cues are displayed at the specific parameters automatically set by the device based on the users recorded gait parameters. For example, Lines, footprints, or other visual cues are spaced to match the step length of the subject. With regards to audio and tactile cues, the audio and tactile cues may be configured to match the subject's walking speed, i.e., cadence measured as steps/min.

The present invention therefore provides for real time feedback based on the subject's observed gait parameters. It has been determined that matching a secondary sensation, i.e., auditory, or tactile, with visual cues can provide an improved response and efficiency in mitigating gait impairment

In one embodiment, a healthcare professional can adjust and set the parameters of the visual/auditory/tactile cues to alter the gait modifying effect of the cue within the game environment. For example, the height of obstacles, spacing between visual floor cues, speed of audio cues etc may be varied.

A subject's response to visual, auditory, and tactile cues may be trained using a game environment. Through observation of the subject's performance in a game, a healthcare professional may adjust certain parameters of the visual, auditory, and tactile cues. For example, if the subject is observed to freeze while performing certain tasks, stronger cues may be presented to the subject either following observation of a freezing event or in response to determining that a freezing event may occur. In contrast, if the subject is observed to not be freezing at all, weaker cues may be presented to the subject. This way, the healthcare professional may vary the cues presented to the subject and seek to challenge the subject in the most appropriate manner.

In one embodiment, a healthcare professional can connect a second AR headset to the subject's session and see the same media content as the subject so that they can guide them through the game and use of the cues within the game environment.

Through use of collaboration software, it is possible for a second person to view what is seen by the subject. This may be in the form of a screenshare, or an overlay that supplement's the second person's own view. Using an AR headset, the second person may audibly guide the subject through a game. The healthcare professional may also interact with game elements, adjust the subject's profile, and place markers to assist the subject in navigating the game.

In one embodiment a user can activate/deactivate cues from UI input through voice command, hand gesture, or controller.

Use of multiple input commands is beneficial where the subject may suffer from impaired cognitive and/or motor function. Providing a range of options increases the likelihood that the subject may be able to interact with the AR headset and the game environment which makes the software accessible for people with various disabilities.

In one embodiment, cues automatically activate/deactivate within a game environment based on the motor state of the user.

As an extension of measuring and recording gait parameters of the subject, systems according to the disclosure may determine a defined motor state of the subject. Depending on the subject's activities within a game environment, particular motor states may be identified as relevant to the subject's current activity. If the system determines that the subject's current motor state matches the target motor state, or is predicted to do so, visual, auditory, or tactile cues may be activated. When the subject's current motor state is then determined not match the target motor state, or is predicted not to match the target motor state, visual, auditory, and tactile cues may be deactivated.

In one embodiment the user can select which visual/auditory/tactile cue they want to assist them within the game environment via the game setting menu from a library of over 100 variations.

The provision of a vast range of visual, auditory, and tactile cues dramatically increases the likelihood of one or more of the stored cues being effective to mitigate gait impairment of the subject. As has already been described, not all cues are effective for all people. Furthermore, an individual may respond differently to different cues in varying circumstances. Therefore, having access to a library of cues is highly advantageous.

In one embodiment, cues are presented in an action relevant way, i.e., if the game environment has a mud surface, then the cues may play a mud sound when stepped on.

By providing consistency between what the subject is seeing through the AR headset and what they can hear and/or feel, consistency between the senses is achieved. It is postulated that such an approach may be highly effective in the alleviation of gait impairments.

In one embodiment the series of transverse bars, images or other visual cues are displayed in different colours depending on the subject's various gait parameters such as walking speed compared to pre-determined threshold walking speeds configured to be associated with respective colours of the series of transverse bars, images, or other visual cues

By measuring and recording the subject's gait parameters, a walking pace, and other characteristics, may be determined. If the user is determined to be walking faster or slower than a target walking pace, the subject may be presented with one or more visual, auditory, or tactile cues to indicate to the subject that their walking pace is suboptimal. By providing such a warning, the subject is provided with the opportunity to vary his/her walking pace.

In one embodiment the augmented reality system is further arranged and adapted to present a series of auditory or tactile cues to the subject at the same time as the series of transverse bars, images or other visual cues are displayed to the subject.

Being able to personalise not only the modality of the cue (Visual, auditory, or tactile) but also the sub-set of that cue is highly important. For example, visual cues can be varied by selecting horizontal bars to step on or obstacles to step over or other type of visual image such as Dinosaur footprints displayed on the floor in a forward pattern which the user matches their own foot placement to when walking. For acoustic cues it could be varying the speed by setting the beats per minute or utilising a library of different audio sound effects from a standard metronome to footsteps on gravel or the sound of high heels on a wooden floor. Visual and acoustic cues can also be combined and used together to further improve personalisation and gait modifying effects of the system.

In one embodiment the augmented reality system is further arranged and adapted to determine the floor surface of the subject's current position; and determining any variation in the floor surface between the subject's current position and the position of the first visual asset and/or any variation in the floor surface between the position of the first visual asset and second visual asset, wherein any one or more of the visual, auditory, and/or tactile visual cues is changeable to coincide with the subject moving from a first floor surface to a second floor surface.

It has been determined that a mismatch of floor surface v sound can contribute towards gait impairment. For example, if a subject is walking on a solid surface, he/she will likely have a loud, prominent footstep. In contrast, if the subject is walking on a carpeted floor, or on the grass outside, it is highly likely that his/her footsteps will be softer and quieter. The present invention facilitates identification of a floor surface through use of the in-headset environmental cameras and sensors and allows for variation of sound played back by the system in response to a varying floor surface.

In one embodiment the augmented reality system is further arranged and adapted to map a physical area surrounding the subject; determine the position of any physical obstacles or hazards within the mapped area; and vary the direction of series of transverse bars, images, or other visual cues to avoid any determined physical obstacles or hazards.

In one example, a game environment may display a series of mole hills to the subject with an in-game character rising out from mole hills at random, or in accordance with a predetermined algorithm. As the mole hills may be generated in any location either inside or outside of the subject's field of vision, it is advantageous to provide visual cues that can guide the subject from his/her current position towards the mole hill. Depending on the environment surrounding the subject, there may be one or more hazards or obstacles that are identified by cameras of the AR headset. Such hazards or obstacles may be mapped in virtual space such that cued path may be varied or adapted to avoid such hazards or obstacles. In some embodiments, the mapped hazards be updated dynamically to take into account moveable objects such as people and pets.

In one embodiment the augmented reality system is further arranged and adapted to place the first and second visual assets in virtual space such that they are presented to the subject within the mapped area.

Ideally, virtual assets will only be displayed to the subject within the mapped area. This facilitates mapping and identification of fixed hazards and obstacles and negates placing of virtual assets in unmapped areas where unknown hazards and obstacles may be present.

In one embodiment the augmented reality system is further arranged and adapted to place at least one intervening visual asset placed between the subject's current position and the position of the first visual asset or second visual asset; and vary the direction of the series of transverse bars, images, or other visual cues to avoid the at least one intervening visual asset.

In a more complex version of the mole hill game environment discussed above, virtual obstacles such as fences, animals, and terrain may be placed within virtual space between the user and a virtual asset. By varying the cued path to avoid such virtual obstacles, a more complex path between the user and the virtual asset is provided.

In one embodiment the augmented reality system is further arranged and adapted to receive an interaction between the subject and the first visual asset and/or second visual asset, wherein such interaction results in the awarding of points or progress towards a points total or progress indicator. In one embodiment the awarded points or progress is variable depending on the speed of travel of the subject from the subject's current position to the first visual asset and/or second visual asset. In one embodiment the awarded points or progress is variable depending on any degree of deviation from a path defined by the series of transverse bars, images, or other visual cues.

One such example might be where the subject stands on a virtual mole rising out of a virtual mole hill. A base points total may be awarded for standing on the mole before a pre-set time runs out. The points total may be increased or decreased based on observed performance metrics such as time taken to complete a task, deviation from a pre-set path, or other parameters.

In one embodiment the series of transverse bars, images or other visual cues is selected for display to the subject depending on a theme of the interactive media asset and/or in response to determining that the subject responds positively to a defined series of transverse bars, images, or other visual cues.

For example, in a game environment where the subject is required to find and smash dinosaur eggs, a series of dinosaur footprints may be generated for display to the subject. Depending on the presented terrain, the subject may also be presented with auditory cues ranging from a simple metronome to mud squelching or gravel crunching. Tactile feedback may also be provided and varied depending on the presented terrain. Furthermore, the subject's response to presented cues may result in selection of different cues to which the subject may be predicted to respond better.

In one embodiment, a rhythmic auditory cue may be activated and dynamically presented to the subject to correspond with the subject's gait speed. In one embodiment, an auditory or tactile response is presented to the user upon the system determining that the user has walked on or over a visual cue.

In addition to displaying a visual cue to a subject, it is beneficial to also present an auditory cue that is matched to the subject's gait speed or cadence. Such an approach provides stimulus through three of the subject's primary senses, i.e., vision, hearing, and feel, and has been determined, in at least some circumstances, to effectively mitigate against gait impairment. A similar effect may be observed through presenting an auditory and/or tactile feedback to the subject upon the subject walking on or over a visual cue.

Further areas of applicability of the present invention will become apparent from the detailed description provided hereinafter. The detailed description and specific examples, while indicating the preferred embodiment of the invention, are intended to be given by way of example only.

### FIGURES

Aspects and embodiments of the invention will now be described by way of reference to the following figures.

FIG. 1 illustrates a system according to the disclosure.

FIG. 2 illustrates a flow chart of a series of steps implemented by an augmented reality system according to the disclosure.

FIG. 3 illustrates a flowchart of a series of steps implemented within an “in-game” environment according to the disclosure.

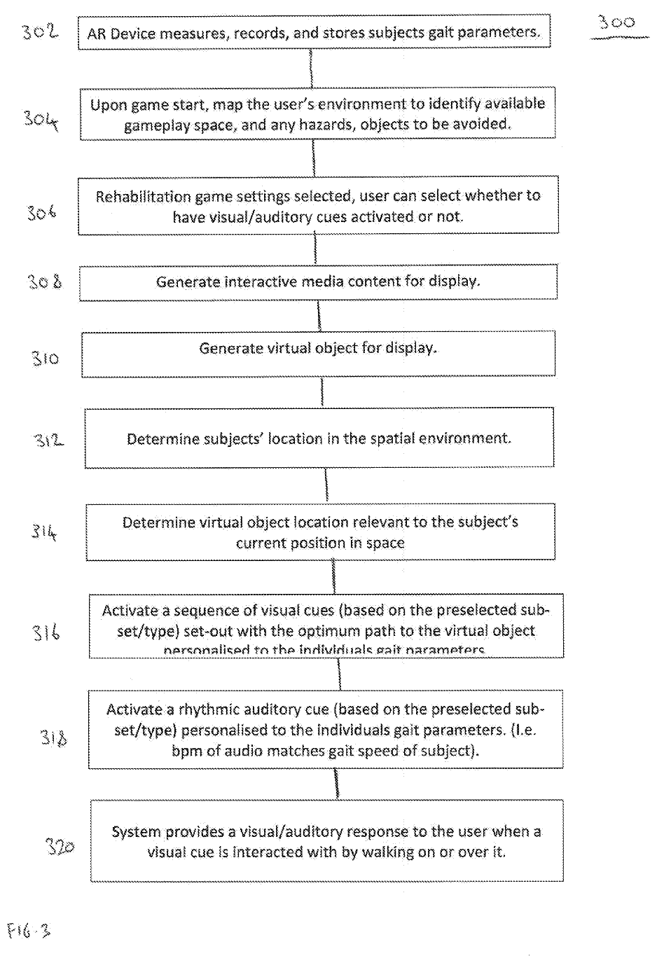

### DESCRIPTION

The following description of the preferred embodiment(s) is merely exemplary in nature and is in no way intended to limit the invention, its application, or uses.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

The applicant has undertaken significant experimentation into presentation of visual, auditory, and tactile cues to subject's suffering from gait impairment because of neurological disorders. As described in the background, it is believed that the presentation of cues to a subject suffering from gait impairment because of neurological disorders causes signals in the brain to be re-routed from damaged or degenerating areas of the brain responsible for automatic motor control to other unaffected/spared brain areas which also have the ability to control motor movement.

It is further believed that the parts of the brain responsible for processing the re-routed signals may be trained to process such signals more effectively. Thus, an augmented reality system is provided according to the claims set out at the end of this document.

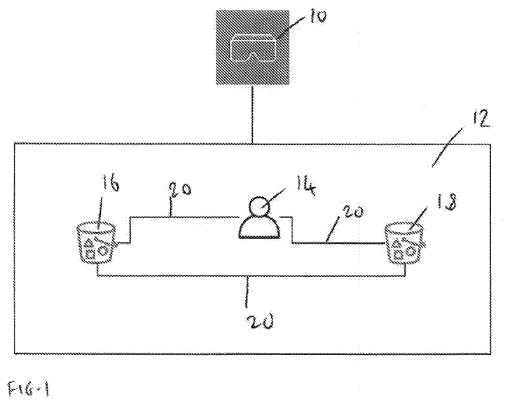

As shown in FIG. 1, an augmented reality system comprises an augmented reality headset 10 adapted and arranged to display interactive content 12 to a subject 14. Within the interactive content 12 at least a first visual asset 16 and second visual asset may be displayed to the subject 14. In between the subject's 14 current position and the position of the first visual asset 16 and second visual asset 18, a series of transverse bars, images, or other visual cues 20 may be displayed or activated. A series of transverse bars, images, or other visual cues 20 may also be displayed between the first visual asset 16 and second visual asset 18. Alongside the visual cues 20, the system may present auditory and/or tactile cues and/or feedback to the subject.

According to the disclosure, the system is arranged and adapted to provide interactive content to the subject. Augmented reality headsets generate holographic images that are generated for display to the subject. Taking the example of therapeutic training content known as “Mole Patrolll”, the system may generate for display one or more mole hills that appear to the subject to be positioned on the floor within a predetermined boundary. The mole hills may be randomly generated for display, or in accordance with a pre-determined pattern. At any one time, one or more characters, i.e., in the form of a mole, may be generated for display in association with respective mole hills. Of course, the actual visual representation of mole hills and moles may be modified to any other suitable visual representation without departing from the scope of the claimed invention. The purpose of such content is to enable the subject to move between mole hills with improved gait and encourage them to stand on the mole. Each time the subject stands on a mole they are awarded points or progress towards a point total or progress indicator.

In another example, the system may generate for display one or more three-dimensional objects that are perceived by the subject to be positioned on a surface or otherwise floating. Again, the objects may be randomly generated for display, or in accordance with a pre-determined pattern. The purpose of this type of content is also to modify and improve the subject's gait pattern and encourage the subject to move between objects and undertake some form of interaction with the object. In an embodiment where a plinth is generated for display with a breakable object, i.e., a vase, positioned atop the plinth, the subject may be encouraged to hit the vase in virtual space to break it and/or knock it off the plinth. The subject may be awarded points or progress towards a point total or progress indicator for each vase they break/knock off.

It will be appreciated that the content types listed above are given by way of example only to explain the context of several embodiments of the inventions and are not intended to limit interpretation of the claims in any way.

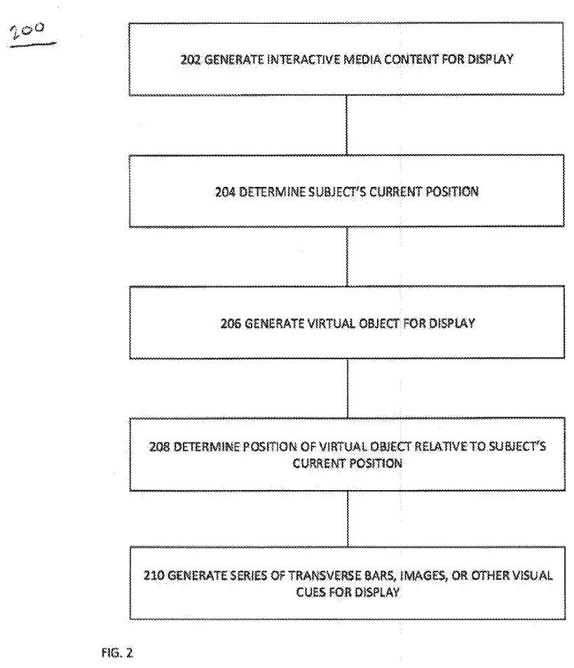

A key feature of the claimed invention is the generation for display of visual, auditory and/or tactile cues to assist the subject to move towards objects displayed within the content and to alleviate gait disturbances/modify gait. This is achieved through the method steps illustrated at FIG. 2 and described below. FIG. 2 illustrates a flow chart of a method 200 implemented by an augmented reality system according to the disclosure. At a first step 202 an interactive media asset is generated for display to the subject by way of an augmented reality device. At a second step 204, the subject's current position is determined by the augmented reality headset. At a third step 206 a first virtual asset, or object, is generated for display to the subject within the interactive content. At a fourth step 208 the position of the first virtual asset, or object, is determined relative to the subject's current position. At a fifth step 210 a series of transverse bars, images, or other visual cues and/or auditory cues and/or tactile cues are generated for presentation to the subject.

An augmented reality or mixed reality headset is a device which permits a person to engage with digital content and interact with digitally superimposed images which are overlaid upon a subject's field of view. The technology may comprise an essentially transparent TFT stereoscopic screen that provides different images to each eye. The headsets may have an array of sensors which sense aspects of the real world using, for example, cameras and/or ultrasonic sensors. The headset preferably includes eye tracking functionality and one or more external cameras. According to the preferred embodiment the subject wears an augmented reality display device which enables the subject to see both real world images which are augmented or enhanced with digitally overlaid visual images. Thus, an interactive media content item may be selected from a library and generated for display to the subject via the augmented reality or mixed reality headset. The augmented reality or mixed reality headset preferably comprises a microphone and natural language processing capability so that voice commands may be received from the subject and/or another person. For example, a voice command to turn visual, auditory or tactile cues on/off may be spoken and interpreted by the augmented reality or mixed reality headset.

One or more gait parameters of the subject may be measured using cameras, IMUs, and other sensors of the augmented reality or mixed reality device and stored in non-volatile memory. The stored gait parameters may be used to optimise the visual, auditory, and tactile cues presented to the subject. For example, the spacing of visual cues may be varied depending on the subject's real time stride length, gait pace and walking speed. The auditory and tactile cues may be varied to match the presented visual cues.

According to the preferred embodiment the augmented reality system is preferably arranged and adapted to determine the subject's current position. For example, the subject's current position may be determined by a global positioning system (“GPS”), a Wi-Fi Positioning System (“WPS”), a geographic information system (“GIS”) or other geospatial locating means. According to another embodiment the subject's current position on a map or layout may also be set or determined by the subject or by a nurse, assistant or practitioner. Once the subject's general geographical position has been determined, the system may map the subject's surroundings using onboard cameras to identify free space and any obstacles and/or hazards. A boundary may then be applied within virtual space with the subject being positioned, at least initially, at the centre of the bounded space.

The subject, patient or an assistant, nurse or practitioner may also use auditory commands to set the location of the subject. For example, the subject or an assistant, nurse or practitioner may verbally tell or otherwise inform the augmented reality system that the subject is starting from a certain location e.g., the lounge or sitting room, or a clinic setting.

One or more virtual assets, or objects, may be generated for display to the subject by way of the augmented reality or mixed reality device. The one or more virtual assets, or objects, may only be generated for display within the bounded area. In some embodiments, the system may determine that there are obstacles or hazards present within the bounded area. In this case, the one or more virtual assets, or objects, will not be placed on, or in the vicinity of, the determined obstacles and/or hazards.

Once the one or more virtual assets, or objects, have been generated for display to the subject, the position of such virtual assets, or objects, is determined relative to the subject. A series of transverse bars, images, or other visual cues and/or auditory cues and/or tactile cues may then be presented to the subject to assist the subject in moving from his/her current position to the position of the one or more virtual assets, or objects. In other words, the series of transverse bars, images, or other visual cues and/or auditory cues and/or tactile cues may act as a stimulated pathway to aid the subject in overcoming gait impairment.

In some embodiments, the system may determine a location of a second of the one or more virtual assets, or objects, and determine the location of said second virtual asset, or object, relative to the first visual asset, or object, and/or the subject's current position. In such an embodiment, the series of transverse bars, images, or other visual cues and/or auditory cues and/or tactile cues may be presented to the subject to assist the subject in moving from a first visual asset, or object, to a second visual asset, or object.

The series of transverse bars, images or other visual cues may be displayed in primary colours. It has been found, for example, to be particularly effective to display the series of transverse or horizontal bars, images or other visual cues in primary colours that alternate red, green, blue, and yellow. Various embodiments are contemplated wherein the series of transverse bars, images or other visual cues may be customised for the subject optionally based upon other subject data and/or after a process of trial and error. The colour and/or sequence of colours used to display the transverse bars, images or other visual cues may be customised. For example, a first subject may find that a sequence of colours red, green, blue then yellow is particularly effective whereas a second subject may find that a different sequence of colours namely red, green, blue, green, yellow, green is particularly effective. A third subject may find that a yet further different sequence of colours red, red, green, green, blue, blue, yellow, yellow is particularly effective. Other embodiments are also contemplated wherein non-primary colours and colours such as white, grey and black may be used.

According to various embodiments the width and/or thickness of the transverse or horizontal bars, images or other visual cues may be arranged to vary, increase, or decrease with distance from the subject. For example, the width of the transverse bars, images or other visual cues may be arranged to progressively increase or decrease with distance away from the current position of the subject. Similarly, the thickness of the transverse bars, images or other visual cues may be arranged to progressively increase or decrease with distance away from the current position of the subject.

It is contemplated that a subject may have a subject profile which includes personalised settings. Aspects such as preferred colours, preferred colour sequence, target step width, target step length may be set or determined in advance and may be stored as a subject profile.

It is also contemplated that the visual cues may take the form of graphical cues such as footprints, rain drops, pebbles, for example. The size, colour, and distance between visual cues, for example, may be varied by the subject or a medical practitioner.

The system may also learn and seek to optimise various parameters during use.

For example, the system may experiment with different colours, different colour sequences, different line widths and/or different line thicknesses and see what settings are optimal for a particular subject. If the system determines that a subject should be moving but no movement is detected, then the system may try different parameters and different combinations of parameters to see whether certain settings elicit a positive response from the subject. Certain parameters of the visual, auditory and tactile cues may be varied in accordance with measured parameters of the subject. For example, if the subject is walking at constant speed the visual cues may be presented in green. Conversely, if the subject is walking erratically then the visual cues may be presented in orange or red depending on the perceived deviation from a baseline walking speed. Similarly, auditory and/or tactile cues may be varied from a spaced apart, gentle cue representation to a close together, energetic cue representation.

For example, the system may display the series of transverse bars, images or other visual cues using default settings but may see whether changes to the default settings are more effective for a particular subject. The system may, for example, initially display the series of transverse bars, images or other visual cues using a default colour sequence of red, green, blue then yellow. However, the system may then experiment and see whether a different sequence of colours red, green, blue, green, yellow, green elicits an improved response from the subject. Similarly, the system may then further test whether another different sequence of colours such as red, red, green, green, blue, blue, yellow, yellow elicits a yet further improved response from the subject.

The series of transverse bars, images or other visual cues may be provided in combination with one or more audio or auditory cues or one or more sound effects. For example, the subject may be of advanced age and hence may have reduced eyesight. Accordingly, the subject may lack confidence to a certain degree in respect of the visual cues which they are provided with. The provision of audio or auditory cues may give the subject greater or additional confidence to step onto the transverse bars, images, or other visual cues.

According to an embodiment the auditory cues may comprise or other auditory effect. the timing of the metronome and/or auditory effect may be customised to match the gait or walking speed of the subject. The metronome or other auditory effect may be accompanied by tactile feedback. In one example, the metronome may be linked to the subject's gait speed and may be presented each time the subject walks on or over a visual cue. Similarly, the tactile feedback may also be presented to the subject each time the subject walks on or over a visual cue.

In some embodiments, cues presented to the user may be evenly spaced apart. In other embodiments, cues presented to the user may be irregularly spaced apart. When different types of cues are used together, i.e., visual and auditory, such cues may be aligned such that sound is presented at the same, or similar, time to a visual presentation. In other embodiments, the sound and visual presentations may be misaligned. Such variations in the presentation of cues allows for flexibility over control over when cues should be presented to the user and may force the subject to consciously adjust their gait for different scenarios.

A step counter may be incorporated to help an assistant or nurse know how far the subject or patient has gone or travelled. For example, the subject may be motivated to perform a certain number of steps over a certain period of time (one day) and the step counter may be linked to other devices such as a Fitbit (RTM) or other personal fitness device in order to feedback to the subject the total number of steps that they have taken over a certain period.

The system preferably includes a spatial data sharing function so that multiple devices can be set up at the same time. Pathways and preferred mapping may be shareable across multiple devices. It is contemplated, for example, that a facility such as a hospital, treatment centre, care home or rest home may make multiple augmented reality headsets, glasses, or other wearable device available to users or patients. The pathways and other geospatial data which may have been initially created for one specific user or patient may be shared more widely within the system so that other users or patients can access the same pathways and geospatial data. The geospatial data may be recorded using a distributed ledger such as blockchain and the system can determine who is accessing the data whilst making sure that the data is only made available to approved devices and practitioners.

The system may include an ID verification routine optionally using biometric data so that a user or patient is uniquely identified before an augmented reality visual overlay is provided to the user or patient. The user or patient may, for example, be identified by iris recognition, facial recognition, or fingerprint recognition. Pathways and maps stored in the blockchain may only be made available to authorised users who optionally have passed an ID verification routine.

The system may include a room detection functionality since the user or patient may not be certain in which room or at which location the user or patient is starting from. Vulnerable patients may need additional support to recognise where they are. A user or patient may use a handheld device or clicker or a voice command to find out which room or location they are in. The user or patient may similarly use the handheld device or clicker or a voice command to request a pathway to an intended or desired destination. According to various embodiments the device may recognise where the user or patient is located, and the user is preferably enabled to ask or click for a pathway to a certain location such as the kitchen without needing to state their current or starting location.

The augmented reality system may include a remote setup function for patients. The remote setup function allows a nurse, assistant, or practitioner to be able to set up the device for the patient in advance which leads to a quicker process in using the system. Furthermore, a nurse, assistant or practitioner is given first-hand experience of using the device so that they can see what the user or patient will see or experience. The nurse, assistant or practitioner when viewing what a user or patient will see or experience can then customise the experience for the user or patient. This is particularly useful when multiple devices are being used in one facility. For example, a facility may stock a plurality of different models of augmented reality headsets or glasses which may have slightly different physical or visual characteristics. Accordingly, a nurse, assistant or practitioner may adjust various parameters of the augmented reality experience dependent upon the model of headset or glasses which the user or patient is to use. According to an embodiment a nurse, assistant or practitioner may be enabled to check the visual set-up of the headset. It has been found, during testing for example, that the view which is provided by the system may be set specific to a particular user or patient. A person setting up the system may be inclined to look more forward than down but this perspective may be different to the preferred visual orientation of the user or patient. Remote set up may be performed by enabling a sharing of headsets and headset views. A phone remote control system APP may be linked between the headset and the patient device via WiFi. Alternatively, a separate headset may be linked to the same networked experience.

The system may be arranged and adapted to track the movement of the user's or patient's hands and/or fingers. In particular, the system may be arranged and adapted to track the movement of the user's or patient's hands and/or fingers in order to determine the rhythm of walking. The same function may also be applied to other parts of the body. For example, the system may track the movement of the arm or other parts of the upper body to monitor whether the user or patient reaches one or more physiotherapy goals which may have been set for them.

The system may also be arranged to provide an augmented visual overlay which colours various objects around them. For example, the floor or carpet on which a user walks may be coloured in a muted colour to minimise potential distraction and to standardise the appearance of the series of transverse bars, images or other visual cues which are preferably displayed over the user's or patient's field of view. Objects which are observed may be recognised using artificial intelligence or machine learning. Objects which have been recognised can be coloured, painted, or otherwise rendered in a standard or user specific manner in the geospatial space. As discussed above, the system may be arranged to provide an augmented reality overlay which visually masks or at least partially obscures any hazards or unmapped areas which might distract, attract, or confuse the user or patient. It has been found that colouring certain objects using an augmented reality visual overlay can help to better coordinate motor responses of users or patients.

The system may determine a difference between floor types, coverings, and colours. For example, if the subject is determined to be likely to move between a carpeted floor and a wooden floor, the system may determine this through use of its on-board cameras. Upon the subject approaching a transition between flooring types, coverings, and colours, the system may vary the of transverse bars, images, or other visual cues and/or auditory cues and/or tactile cues, to differentiate between the different flooring types, coverings, and colours. For example, a series of dark coloured visual cues may be generated for display via the augmented reality or mixed reality device when such visual cues are to be projected on to a light surface. Conversely, a series of light-coloured visual cues may be generated for display via the augmented reality or mixed reality device when such visual cues are to be projected on to a dark surface. Furthermore, the shape, thickness, category, spacing difference etc may be varied according to the determined flooring type, covering, or colour. Such customization of visual cues may be personal to the subject. In addition, any auditory or tactile cue being presented to the subject may also be varied in accordance with a change in flooring type, covering, or colour. For example, the sound generated while a subject is working on a carpeted floor may be different to the sound that is generated while a subject is walking on a wooden floor. Similarly, the tactile feedback provided for different flooring types, coverings, or colours may be varied according to subject preference and/or as determined by a medical professional. Cues may be selected to be presented permanently, in response to a predicted motor state of the subject, or not at all.

In some embodiments, the system may determine that there are one or more intervening obstacles, or hazards, between the subject's current position and the position of a virtual asset, or object. The series of transverse bars, images, or other visual cues may thus be redirected to avoid the perceived intervening obstacles, or hazards.

In another embodiment, a method 300 starts at step 302. At step 302, an augmented reality headset is configured to measure, record, and store predetermined gait parameters of a subject. For example, the subject's gait speed, stride length, and stride variability may be measured, recorded, and stored. At step 304 a game may be launched via the augmented reality headset and the subject's environment may be mapped using cameras and other sensors of the augmented reality headset. Following the mapping, the available gameplay space may be identified and any hazards and obstacles pinpointed. At step 306 required game settings, i.e., rehabilitation settings, are selected together with whether cues should be activated at the start of the game, or not. At step 308, the game is generated for display via the augmented reality headset. At step 310 a virtual object is generated for display via the augmented reality headset. At step 312 the subject's location in the mapped/spatial environment is determined. At step 314 the location of the virtual object relative to the subject's location is determined. At step 316 a cue path comprising visual cues is generated for display via the augmented reality headset between the subject's determined position and the position of the virtual object. At step 318 a rhythmic auditory cue is presented to the subject and configured to match one or more gait parameters of the subject. At step 320 a visual, auditory, or tactile response is presented to the subject when he/she walks on or over a visual cue. For example, the response could be a changing of the visual cue colour, presenting a sound, or vibration of the augmented reality headset.

As referred to herein, processing circuitry should be understood to mean circuitry based on one or more microcontroller units, digital signal control units, programmable logic devices, field-programmable gate arrays (FPGAs), application-specific integrated circuits (ASICs), etc. and may include a multi-core control unit (e.g., dual-core, quad-core, hexa-core, or any suitable number of cores). In some embodiments processing circuitry may be distributed across multiple separate control units or processing units, for example multiple of the same type of processing units (e.g., two Intel Core i7 control units) or multiple different control units (e.g., an Intel Core i5 control unit and an Intel Core i7 control unit). In some embodiments, processing circuitry executes instructions for receiving sensor data and applying FES to a subject, wherein such instructions are stored in non-volatile memory.

In client-server-based embodiments, processing circuitry may include communication means suitable for communication with an external computing device server or other networks or servers. The instructions for carrying out the above-mentioned functionality may be stored in the non-volatile memory or on the external computing device. Processing circuitry may include a cable modem, an integrated services digital network (ISDN) modem, a digital subscriber line (DSL) modem, a telephone modem, Ethernet card, or a wireless modem for communications with other equipment, or any other suitable communications circuitry such as WiFi or Bluetooth components. Such communications may involve the Internet or any other suitable communications networks or paths. In addition, communications means may include circuitry that enables peer-to-peer communications of external computing devices, or communication of external computing devices, or communication of external computing devices in locations remote from each other.

Non-volatile memory may be embodied in an electronic storage device that is part of processing circuitry. As referred to herein, the phrase “electronic storage device” or “storage device” should be understood to mean any device for storing electronic data, computer software, or firmware, such as random-access memory, read-only memory, hard drives, flash drives, SD cards, for example.

It should be appreciated that in the above description of exemplary embodiments, various features are sometimes grouped together in a single embodiment, figure, or description thereof for the purpose of streamlining the disclosure and aiding in the understanding of or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

While some embodiments described herein include some, but not other features included in other embodiments, combinations of features of different embodiments are meant to be within the scope of the disclosure, and form different embodiments, as would be understood by the skilled person. For example, in the following claims, any of the claimed embodiments can be used in any combination.

Thus, while certain embodiments have been described, it will be appreciated that other and further modifications may be made thereto without departing from the spirit of the disclosure, and it is intended to cover all such modifications, enhancements, and other implementations, which fall within the true spirit and scope of this disclosure. To the maximum extent permitted by law, the scope of this disclosure is to be determined by the broadest permissible interpretation of the following claims, and shall not be restricted or limited by the foregoing detailed description

While various implementations of the disclosure have been described, it will be readily apparent to the skilled person that many more implementations are possible within the scope of the disclosure.

1. An augmented reality system for presenting cues within an augmented reality game environment to a subject who experiences gait impairment, wherein the augmented reality system comprises a display for providing an augmented reality overlay over a subject's field of vision and wherein the augmented reality system is arranged and adapted to:
generate for display to the subject an interactive media asset;
determine the subject's current position;
generate for display to the subject a first virtual asset;
determine the position of the first virtual asset relative to the subject's current position; and
generate for display to the subject at least one cue which acts to prompt the subject to step forwards and walk from the subject's current position to the position of the first virtual asset.

2. The augmented reality system of claim 1, wherein at least one cue comprises a series of transverse bars, images or other visual cues which appear to the subject to be located on the ground in front of the subject to step on or over.

3. The augmented reality system of claim 1, wherein the augmented reality display comprises a headset or glasses.

4. The augmented reality system of claim 1, further arranged and adapted to:
generate for display to the subject a second virtual asset; determine the position of the second virtual asset relative to the first virtual asset and/or subject's current position; and
generate for display to the subject a series of transverse bars, images or other visual cues which appear to the subject to be located on the ground in front of the subject and which act to prompt the subject to step forwards and follow the transverse bars, images, or other visual cues from the position of the first virtual asset to the position of the second virtual asset.

5. The augmented reality system of claim 2, wherein the
series of transverse bars, images or other visual cues is displayed in different colours depending on the subject's current position relative to the position of the series of transverse bars, images, or other visual cues

6. The augmented reality system of claim 5, wherein the series of transverse bars, images, or other visual cues is displayed in different colours depending on the subject's walking speed compared to pre-determined threshold walking speeds configured to be associated with respective colours of the series of transverse bars, images, or other visual cues

7. The augmented reality system of claim 2 further comprising presenting a series of auditory or tactile visual cues to the subject at the same time as the series of transverse bars, images or other visual cues are displayed to the subject.

8. The augmented reality system of claim 7 further comprising:
determining the floor surface of the subject's current position; and
determining any variation in the floor surface between the subject's current position and the position of the first visual asset and/or any variation in the floor surface between the position of the first visual asset and second visual asset,
wherein any one or more of the visual, auditory, and/or tactile visual cues is changeable to coincide with the subject moving from a first floor surface to a second floor surface.

9. The augmented reality system of claim 2 further comprising:
mapping a physical area surrounding the subject;
determining the position of any physical obstacles or hazards within the mapped area; and
varying the direction of series of transverse bars, images, or other visual cues to avoid any determined physical obstacles or hazards.

10. The augmented reality system of claim 9 further comprising placing the first and second visual assets in virtual space such that they are presented to the subject within the mapped area.

11. The augmented reality system of claim 10 further comprising:
placing at least one intervening visual asset placed between the subjects' current position and the position of the first visual asset or second visual asset; and
varying the direction of the series of transverse bars, images or other visual cues to avoid the at least one intervening visual asset.

12. The augmented reality system of claim 1 further comprising receiving an interaction between the subject and the first visual asset and/or second visual asset, wherein such interaction results in the awarding of points or progress towards a point total or progress indicator.

13. The augmented reality system of claim 11, wherein the awarded points or progress is variable depending on the speed of travel of the subject from the subject's current position to the first visual asset and/or second visual asset.

14. The augmented reality system of claim 11, wherein the awarded points or progress is variable depending on any degree of deviation from a path defined by the series of transverse bars, images, or other visual cues.

15. The augmented reality system of claim 1, wherein the series of transverse bars, images or other visual cues is activated in response to a command from the subject or another person.

16. The augmented reality system of claim 2, wherein the series of transverse bars, images or other visual cues is selected for display to the subject depending on a theme of the interactive media asset and/or in response to determining that the subject responds positively to a defined series of transverse bars, images, or other visual cues.

17. The augmented reality system of claim 1 further comprising means for measuring and recording gait parameters of the subject.

18. The augmented reality system of claim 17, wherein one or more cues are presented according to pre-set parameters based on the subject's recorded gait parameters.

19. The augmented reality system of claim 18, wherein the subject or another person may adjust and set predefined parameters of the visual, auditory or tactile cues to alter a target gait modifying effect provided by the visual, auditory or tactile cue within the game environment.

20. The augmented reality system of claim 19, wherein the subject or another person can activate/deactivate cues from UI input through voice command, hand gesture, or controller; wherein, cues are configured to automatically activate/deactivate within a game environment based on the determined motor state of the subject; and wherein the subject can select which visual/auditory/tactile cue they want to assist them within the game environment via a library of cues; and wherein cues are presented in an action relevant way.

21. (canceled)

22. (canceled)

23. (canceled)
